(19) United States
(12) Patent Application Publication
(10) Pub. No.: US 2008/0262328 A1
(43) Pub. Date: Oct. 23, 2008

(54) Pulse Oximetry Grip Sensor and Method of Making Same

(75) Inventor: Bradley Adams, W. Leechburg, PA (US)

Correspondence Address: GREGORY L BRADLEY;MEDRAD INC, ONE MEDRAD DRIVE, INDIANOLA, PA, 15051, US

(73) Assignee: MEDRAD, INC., Indianola, PA (US)

(21) Appl. No.: 11/814,496

(22) PCT Filed: Jan. 23, 2006
(86) PCT No.: PCT/US2006/002384

§ 371 Date: Jul. 23, 2007

Related U.S. Application Data
(60) Provisional application No. 60/646,207, filed on Jan. 21, 2005.

(51) Int. Cl. A61B 5/1455

(52) U.S. Cl. 600/344; 425/542; 264/1.7

(57) ABSTRACT

A probe for use with a pulse oximeter apparatus is disclosed, as are a fixture for and a method of making the probe. The probe includes a housing and two fiber optic bundles. The first bundle has an emitter portion at one end, and is used for conducting light from a source thereof to the emitter portion from which the light is transmitted for transillumination through a body part. The second bundle is for conducting the transilluminated light incident upon a detector portion thereof to the pulse oximeter apparatus. The housing is overmolded onto the bundles so that the emitter and detector portions are securely positioned diametrically opposite each other across an opening defined by the housing. The overmolding makes the alignment of the emitter and detector portions largely impervious to movement of the body part when placed within the opening and thus between the emitter and detector portions.

### FIELD OF THE INVENTION

The invention relates generally to mechanisms for monitoring the extent to which arterial blood of a patient is saturated with oxygen, and more particularly to probes of the type whose fiber optic bundles are used with pulse oximeters to achieve that goal. Even more particularly, the invention pertains to probes of the type that clamp, clasp or otherwise couple to a body part for the purpose of holding the emitter and detector portions of the fiber optic bundles onto the body part and routing the relevant light signals between the body part and such pulse oximeters.

### BRIEF DESCRIPTION OF RELATED ART

The following information is provided to assist the reader to understand the invention disclosed below and at least some of the applications in which the invention will typically be used. In addition, any references set forth herein are intended merely to assist in such understanding. Inclusion of a reference herein, however, is not intended to constitute an admission that it is available as prior art against the invention.

Pulse oximetry is a well known procedure used to measure the degree to which the blood of a patient is saturated with oxygen. Using pulse oximetry to measure the oxygen level (also referred to as “oxygen saturation”) in the blood is considered to be a noninvasive, painless way of providing a general indication how well oxygen is being delivered to the tissues of the body.

Oxygen saturation is a measure of how much oxygen the blood is carrying as a percentage of the maximum it can carry. With each breath, oxygen is passed by the lungs to the blood stream where the majority of the oxygen attaches to hemoglobin. Hemoglobin is a protein located inside each red blood cell, and the hemoglobin molecules in blood are what carry oxygen from the lungs to the tissues of the body and return carbon dioxide from the tissues to the lungs for re-exchange with oxygen. One hemoglobin molecule can carry a maximum of four molecules of oxygen. If a hemoglobin molecule is carrying three molecules of oxygen, then it is carrying ¾ths or 75% of the maximum amount of oxygen that it can carry. One hundred hemoglobin molecules can together carry a maximum of 400 (100×4) oxygen molecules. If those 100 hemoglobin molecules were carrying 380 oxygen molecules, they then would be carrying (380/400)×100=95% of the maximum number of oxygen molecules that they can carry and so together would be 95% saturated. In healthy patients, a normal oxygen saturation level is typically around 97-98 percent.

Pulse oximetry technology takes advantage of the light absorptive characteristics of hemoglobin and the pulsating nature of blood flow in the arteries to aid in determining the oxygen saturation of the blood in the body. First, there is a color difference between hemoglobin that is fully saturated with oxygen and hemoglobin with little or no oxygen bound to it, with the former being bright red and the latter being much darker. Second, with each pulse or heartbeat there is a slight increase in the volume of blood flowing through any given artery or branch thereof. Because of this increase in blood volume, albeit small, there is a corresponding increase in oxygen-rich hemoglobin. Each pulse essentially represents the maximum amount of oxygen-rich hemoglobin flowing through the arterial vessels at any given time.

Pulse oximetry systems used to measure the oxygen saturation of blood typically include a probe and a computerized system (often referred to as a pulse oximeter apparatus) to which the probe connects. Sometimes embodied in the form of a clip, the probe is designed to be clamped, clasped, taped or otherwise coupled to a body part (e.g., a finger, an earlobe, or a nose). For use in magnetic resonance (MR) applications, oftentimes such probes generally consist of two fiber optic bundles and a coupling member. The coupling member is used to hold the emitter and detector portions of the fiber optic bundles onto the body part so that the emitter and detector portions are aimed at each other therethrough. As is explained below, the probe via its fiber optic bundles is also used to route the relevant light signals between the body part and the pulse oximeter apparatus. The pulse oximeter apparatus itself typically includes a transmitter unit, a receiver unit, and a microprocessor through which the measurement of oxygen saturation of the blood is ultimately made and controlled.

When the probe is connected to the pulse oximeter apparatus, one of its fiber optic bundles is optically coupled to two light-emitting diodes (LEDs) or other suitable light source(s) within the transmitter unit from which the bundle receives light of two different wavelengths. One of these wavelengths is chosen from the red band (typically 650 to 670 nm), and the other from the infrared band (typically 920 to 960 nm). The other fiber optic bundle of the probe is optically coupled to a photodetector (e.g., a photo diode or phototransistor) within the receiver unit. The photodetector is sensitive to the return light signals received from the detector portion of the return fiber optic cable. Typically consisting of numerous (e.g., 200-400) fibers, each fiber optic bundle will have its ends ground and polished to make the transfer of light efficient as possible between, for example, the light source(s) and the emitting fiber optic bundle and between the return fiber optic bundle and the photodetector(s) of the receiver unit.

In operation, a pulse oximetry system will emit the two wavelengths of monochromatic light (e.g., 660 nm and 940 nm) from its light source into one end of the emitting fiber optic bundle. As a waveguide, the fiber optic bundle conducts the transmitted light to its emitter portion at its other end from which the light is transilluminated through the body part (e.g., finger) on which the coupling member is mounted. As the light passes through the body part to the detector portion of the return fiber optic bundle, the oxygen-rich hemoglobin in the arterial vessels of the body part tend to absorb more of the infrared light and the oxygen-depleted hemoglobin absorbs more of the red light. The transilluminated light is then detected by the detector portion and conveyed by the return fiber optic bundle to the receiver unit of the pulse oximeter apparatus. From the return light signals supplied by the receiver unit, the microprocessor is also capable of distinguishing pulsatile blood flow from other more static signals (such as tissue or venous signals). This enables the pulse oximetry system to calculate the oxygen saturation using the blood flowing through the arterial capillary bed of the body part rather than venous vessels.

Using the pulsatile blood flow, the microprocessor of the pulse oximeter apparatus calculates how much infrared light has been absorbed versus the amount of red light absorbed. The ratio of this pulse-added red absorbance to the pulse-added infrared absorbance is used to produce a measurement called the spot oxygen saturation level or SpO2, which is an estimate of the actual oxygen saturation level of arterial blood or SaO2. In calculating the SpO2 level, the microprocessor takes advantage of previously determined calibration curves that relate transcutaneous light absorption to direct SaO2. The microprocessor then displays the SpO2 level (i.e., the percentage of hemoglobin saturated with oxygen) and pulse rate, and, in some models, a graph indicative of the quality of the blood flow. Audible alarms are also provided on many pulse oximetry systems. Often programmable, such alarms can provide an audible signal for each heartbeat and, more importantly, audible warnings of hypoxia (low oxygen level) before the patient becomes clinically cyanosed (blue discoloration of tissue due to deficiency of oxygen). Overall, pulse oximetry systems help medical personnel assess the amount of oxygen being carried in the blood and evaluate the need for supplemental oxygen.

The probes offered with many commercially available pulse oximetry systems exhibit significant shortcomings in design, and as a result have proven somewhat labor intensive and time consuming to use. Many of these probes feature a coupling member having slots or notches on opposite sides of the opening into which the body part is designed to be inserted and held. The emitter and detector portions of the fiber optic cables are designed to snap-fit or otherwise fasten into these slots so that they face each other across the opening. If the emitter and detector portions are either not oriented properly within the notches or not inserted into the proper slots, the pulse oximetry system will provide inaccurate SpO2 measurements or fail to provide such measurements at all. For example, U.S. Pat. No. 5,786,592 to Hök, incorporated herein by reference, discloses two fiber optic cables whose distal ends are bent and inserted within upper and lower parts of a clamp-like probe assembly. The upper and lower parts of the probe are connected via a pivot assembly and are held normally closed via a spring-like elastic ring. Similarly, U.S. Pat. No. 5,279,295 to Martens et al., also incorporated herein by reference, discloses two light waveguides that mount into corresponding plugs within upper and lower parts of a clamp-like probe assembly. In each of these prior art probes, the alignment of the emitter and detector portions depends not only on proper assembly of the upper and lower parts of the clamp but also on the proper placement of the emitter and detector portions within the upper and lower parts, respectively, of such clamp-like probe assemblies.

The MRI SpO2 Grip Sensor™ offered by Invivo Research, Inc. also requires the assembly of fiber optic cables to a coupling member. As disclosed in Brochure No. LL149 Rev B, the Grip Sensor™ features a coupling member, which is referred to as a grip, and two fiber optic cables. As with probes made by other manufacturers, the coupling member of the Grip Sensor™ is offered in different sizes (e.g., neonatal, infant, pediatric/small adult and adult sizes). Referred to as fiber optic buttons, the emitter and detector portions of the cables are designed to snap-fit into the grip via two slots defined therein on opposite sides of the opening for the body part. The brochure warns, however, that if the buttons are not inserted and oriented properly within the slots, the Grip Sensor™ will not allow the pulse oximeter apparatus with which it is used to provide an SpO2 reading and an error message will be displayed as a result.

Each of the above references therefore discloses a probe in which the means and method of alignment of the emitter and detector portions pose a burden on the customer. For the preassembled probes taught in the '592 and '295 patents, the customer is required to assure that the emitter and detector portions of the fiber optic cables are properly aligned within the upper and lower parts, respectively, of a clamp-like coupling member. The customer must also make sure that the upper and lower parts of the clamp are themselves properly aligned relative to each other. For the Grip Sensor™ probe offered by Invivo Research, the customer is required to assemble the buttons (emitter and detector portions) into the grip (coupling member) in addition to assuring that they are properly aligned. Furthermore, the manner in which the emitter and detector portions are held within these coupling members—whether manifested as separate and aligned parts or in slots, notches or otherwise in a single-piece coupling member—leaves them susceptible to becoming misaligned due to movement of the patient. Misalignment whether due to issues of probe design or susceptibility to patient movement not only gives rise to inaccurate SpO2 readings and provokes the concern and attention of medical personnel but also imposes undue labor upon such personnel—and its inevitable costs—in tracking down its source.

It is therefore desirable to develop a pulse oximetry probe whose emitter and detector portions are securely held by a coupling member so that they are aligned diametrically opposite each other across the opening defined by the coupling member. The coupling member would preferably be manifested as a single-piece overmolded onto the emitter and detector portions. The overmolding method is desirable because the alignment of the emitter and detector portions accomplished thereby would be made largely impervious to movement of the body part when the body part is placed within the housing and thus between the emitter and detector portions secured therein.

### SUMMARY OF THE INVENTION

Several objectives and advantages of the invention are attained by the preferred and alternative embodiments and related aspects of the invention summarized below.

In a first embodiment, the invention provides a probe for use with a pulse oximeter apparatus. The probe includes a first fiber optic bundle, a second fiber optic bundle, and a housing. The first fiber optic bundle has an emitter portion at one end thereof. The first fiber optic bundle is used for conducting light from a source thereof to the emitter portion from which the light is transmitted for transillumination through a body part. The second fiber optic bundle has a detector portion at one end thereof. The second fiber optic bundle is used for conducting the transilluminated light incident upon the detector portion to the pulse oximeter apparatus. The housing is overmolded onto the first and second fiber optic bundles so that the emitter and detector portions thereof are securely positioned diametrically opposite each other across an opening defined by the housing. The overmolding makes the alignment of the emitter and detector portions largely impervious to movement of the body part when the body part is placed within the opening of the housing and thus between the emitter and detector portions therein.

In a related aspect, the invention also provides a fixture for making a probe for a pulse oximeter apparatus. The fixture includes a base member, an opposing member, and an insert member. The insert member is intended for placement between the base and opposing members. The members together define a cavity of predetermined shape wherein a housing of the probe is formable via introduction of a flowable material therein via at least one conduit defined by at least one of the members. The members further define two channels for holding first and second fiber optic bundles, respectively, as the housing is overmolded thereabout so that an emitter portion of the first fiber optic bundle and a detector portion of the second fiber optic bundle are securely positioned diametrically opposite each other across an opening defined by the housing. The overmolding of the fiber optic bundles makes the alignment of the emitter and detector portions largely impervious to movement of a body part when the body part is positioned within the opening of the housing and between the emitter and detector portions therein during use of the probe.

In a further related aspect, the invention also provides a method of making a probe for use with a pulse oximeter apparatus. The method includes the following steps: (a) positioning a pair of fiber optic bundles on an insert member of a mold so that an emitter portion of a first of the fiber optic bundles and a detector portion of a second of the fiber optic bundles are positioned diametrically opposite each other a predetermined distance apart; (b) placing the insert member on which the emitter and detector portions have been positioned upon a base member of the mold; (c) placing an opposing member of the mold onto the insert member, with the members together defining a cavity of predetermined shape wherein a housing of the probe is formable via introduction of a flowable material therein; and (d) introducing the flowable material into the cavity to at least partially overmold the fiber optic bundles therein and form the housing thereabout so that the emitter and detector portions are securely positioned diametrically opposite each other across an opening defined by the housing. The overmolding of the two fiber optic bundles makes the alignment of the emitter and detector portions largely impervious to movement of a body part when the body part is positioned within the opening of the housing during use of the probe.

In another related aspect, the invention provides a method of making a probe for use with a pulse oximeter apparatus. The method includes the following steps: (a) attaching a base member of a mold to a stationary platen of a molding system; (b) attaching an opposing member of the mold to a movable platen of the molding system; (c) positioning a pair of fiber optic bundles on an insert member of the mold so that an emitter portion of a first of the fiber optic bundles and a detector portion of a second of the fiber optic bundles are positioned diametrically opposite each other a predetermined distance apart; (d) placing the insert member on which the emitter and detector portions have been positioned upon the base member of the mold; (e) pressing the opposing member onto the insert member via the movable and stationary platens of the molding system, with the members together defining a cavity of predetermined shape wherein a housing of the probe is formable via introduction of a flowable material therein; and (f) introducing the flowable material into the cavity to at least partially overmold the fiber optic bundles therein and form the housing thereabout so that the emitter and detector portions are securely positioned diametrically opposite each other across an opening defined by the housing. The overmolding makes the alignment of the emitter and detector portions largely impervious to movement of a body part when positioned within the opening of the housing and thus between the emitter and detector portions therein during use of the probe.

In a second embodiment, the invention provides a probe for use with a pulse oximeter apparatus. The probe includes a first fiber optic bundle, a second fiber optic bundle, an emitter subhousing, a detector subhousing, and a housing. The first fiber optic bundle has an emitter portion at one end thereof, and is used for conducting light from a source thereof to the emitter portion from which the light is transmitted for transillumination through a body part. The second fiber optic bundle has a detector portion at one end thereof. The second fiber optic bundle is used for conducting the transilluminated light incident upon the detector portion to the pulse oximeter apparatus. The emitter portion of the first fiber optic bundle is inserted into the emitter subhousing, and the detector portion of the second fiber optic bundle is inserted into the detector subhousing. The housing is overmolded onto the emitter and detector subhousings so that the emitter and detector portions inserted therein, respectively, are securely positioned diametrically opposite each other across an opening defined by the housing. The overmolding makes the alignment of the emitter and detector portions largely impervious to movement of the body part when placed within the opening and between the emitter and detector portions therein.

In a related aspect, the invention also provides a fixture for making a probe for a pulse oximeter apparatus. The fixture includes a base member, an opposing member, and an insert member. The insert member is intended for placement between the base and opposing members. The members together define a cavity of predetermined shape wherein a housing of the probe is formable via introduction of a flowable material therein via at least one conduit defined by at least one of the members. The members further define two channels for holding first and second fiber optic bundles, respectively, as the housing is overmolded about both an emitter subhousing and a detector subhousing into which an emitter portion of the first fiber optic bundle and a detector portion of the second fiber optic bundle have been respectively inserted. The emitter and detector portions are securely positioned diametrically opposite each other across an opening defined by the housing thereby making the alignment of emitter and detector portions largely impervious to movement of a body part when the body part is positioned within the opening and between the emitter and detector portions therein during use of the probe.

In a further related aspect, the invention also provides a method of making a probe for use with a pulse oximeter apparatus. The method includes the following steps: (a) inserting an emitter portion of a first fiber optic bundle into an emitter subhousing; (b) inserting a detector portion of a second fiber optic bundle into a detector subhousing; (c) positioning the emitter and detector subhousings on an insert member of a mold so that the emitter portion of the first fiber optic bundle and the detector portion of the second fiber optic bundle are positioned diametrically opposite each other a predetermined distance apart; (d) placing the insert member on which the emitter and detector subhousings have been positioned upon a base member of the mold; (e) placing an opposing member of the mold onto the insert member, with the base, insert and opposing members together defining a cavity of predetermined shape wherein a housing of the probe is formable via introduction of a flowable material therein; and (f) introducing the flowable material into the cavity to at least partially overmold the emitter and detector subhousings therein thereby forming the housing thereabout so that the emitter and detector portions are securely positioned diametrically opposite each other across an opening defined by the housing so that an alignment of the emitter and detector portions is made largely impervious to movement of a body part when the body part is positioned within the opening of the housing during use of the probe.

### BRIEF DESCRIPTION OF THE DRAWINGS

The invention will be better understood by reference to the detailed disclosure below and to the accompanying drawings, in which:

FIG. 1 illustrates a perspective view of a pulse oximetry probe according to a first embodiment of the invention.

FIG. 2 illustrates a top view of the pulse oximetry probe of FIG. 1.

FIG. 3 illustrates a cross-sectional side view of the pulse oximetry probe of FIG. 1.

FIG. 4 illustrates a front view of the pulse oximetry probe of FIG. 1.

FIG. 5 illustrates a top view of the housing of the pulse oximetry probe of FIG. 1.

FIGS. 6A-6F illustrate various states of assembly of the insert, base and opposing members of a fixture for enabling production of the pulse oximetry probe of FIG. 1.

FIG. 7 illustrates an enlarged perspective view, from the rear, of a pulse oximetry probe according to a second embodiment of the invention.

FIG. 8 illustrates an enlarged perspective view of the housing, and subhousings overmolded thereby, of the pulse oximetry probe of FIG. 7.

FIG. 9 illustrates the subhousings of the pulse oximetry probe of FIGS. 7 and 8.

FIG. 10 illustrates a subfixture for making the subhousings of FIGS. 7-9.

FIGS. 11A-11G illustrate various states of assembly of the insert, base and opposing members of a fixture for enabling production of the pulse oximetry probe of FIG. 7.

### DETAILED DESCRIPTION OF THE INVENTION

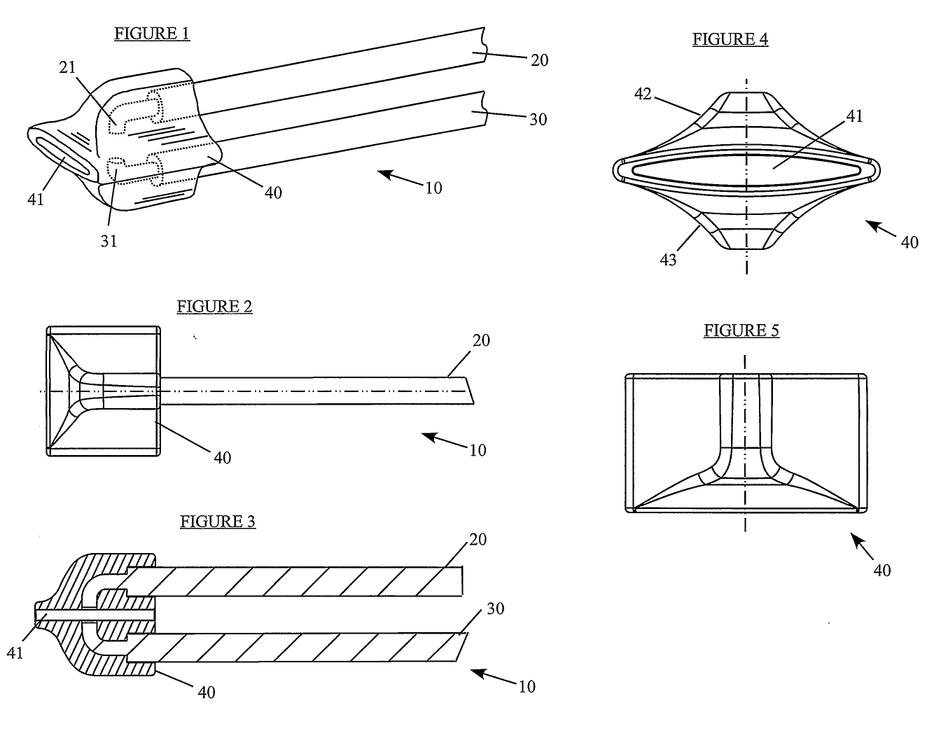

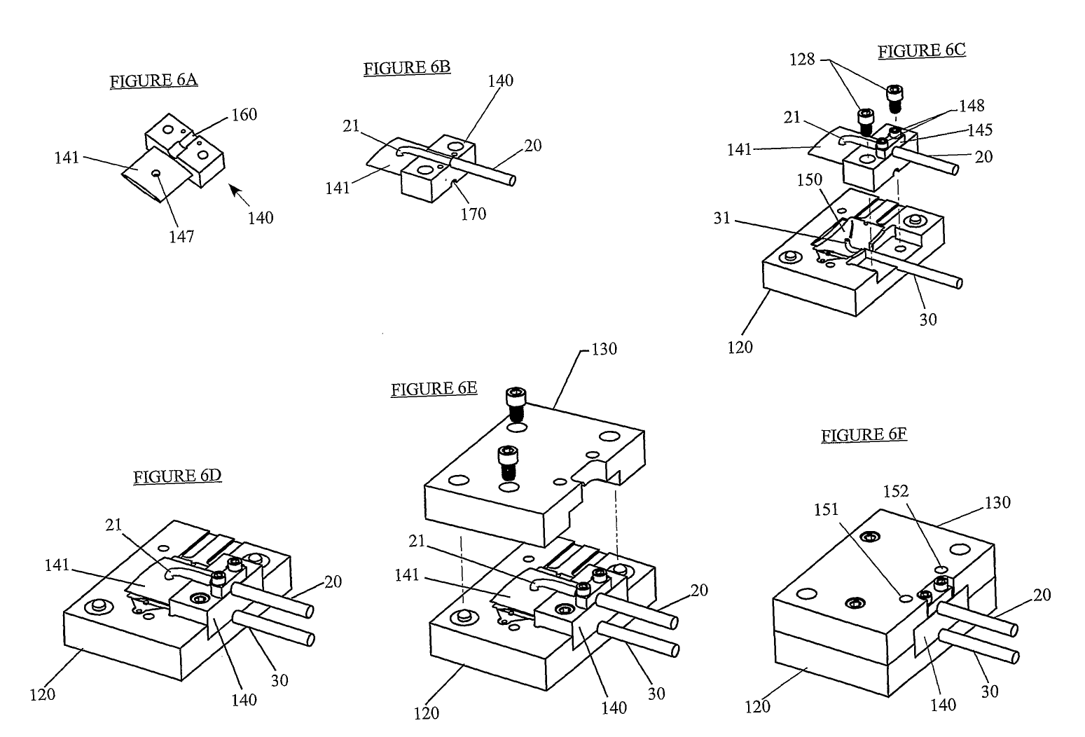

FIGS. 1-5 illustrate a probe for use with a pulse oximeter apparatus, and FIGS. 6A-6F illustrate a mold fixture for making such a probe. These figures and the following detailed description also provide the essential details for a method of making the probe.

FIGS. 1-5 illustrate the pulse oximetry probe made according to a first embodiment of the invention. The probe, generally designated 10, includes two fiber optic bundles 20 and 30 and a housing 40. Typically consisting of numerous fibers, each fiber optic bundle will preferably have its ends ground and polished to make the transfer of light efficient as possible. In this regard, the first fiber optic bundle 20 features an emitter portion 21 at one of its ends. As best shown in FIGS. 1 and 3, this end will preferably be right-angled so that it may be easily positioned upon an insert member of a mold as will be explained below. Similarly, the second fiber optic bundle 30 includes a detector portion 31 at one of its ends, and it too will preferably be right-angled so that it may be easily positioned upon an opposite side of the insert member. The first fiber optic bundle 20 is used for conducting light from the pulse oximeter apparatus (not shown) or other light source to the emitter portion 21 from which the light is transmitted for transillumination through a body part. For ease of explanation, the body part will hereinafter be referred to as a finger, even though other body parts (e.g., earlobe and nose as well as the feet and hands of neonatal patients, etc.) will enable the invention to operate as intended.

The second fiber optic bundle 30 is used for conducting the transilluminated light incident upon the detector portion 31 to the pulse oximeter apparatus. More specifically, the transilluminated light is received by the detector portion 31 and conveyed by the second fiber optic bundle 30 to a photodetector (not shown) or similar receiver unit of the pulse oximeter apparatus. Using the transilluminated light along with the pulsatile blood flow, the pulse oximeter apparatus is then able to calculate the SpO2 level according to well known techniques.

In several respects, the fiber optic bundles of the invention may be manifested in any number of known constructions or variations thereon. Each fiber optic bundle may, for example, include approximately 3000 optical fibers, each of which having a diameter of approximately 50 microns. Moreover, the ends of the fibers are preferably glued together, for example by epoxy, and then polished to form the respective emitter and detector portions. The emitter and detector portions 21 and 31 are then preferably coated with a layer of optically clear silicone, which serves to protect the ends yet still allow for the efficient transfer of light. Each bundle of optical fibers should be protected with a suitable covering such as a polyethylene material, and will preferably be approximately 0.125 inches in diameter. Except for their ends, the two fiber optic bundles 20 and 30 may themselves be bound within a single sheath to facilitate handling.

At the ends opposite the emitter and detector portions, the fiber optic bundles 20 and 30 may be implemented in whatever manner the pulse oximeter apparatus with which they are to be used requires. For example, the ends may be optoelectrically connected to electrical wires via phototransducers (e.g., phototransistors and photodiodes). These two electrical wires at their other ends may be incorporated into a male Lemo® connector through which they can be connected via a female Lemo® connector to the transmitter and receiver units of the pulse oximeter apparatus. This is similar to the interconnection scheme employed on the 3500 Pulse Oximetry Monitor or the 9500 Multigas Monitor, incorporated herein by reference, produced by MEDRAD, Inc, of Indianola, Pa. When the probe of the invention is to be used in the scanner room of an MRI suite, the section of the sheathed probe cable having the phototransducers and electrical wires—or at least that part of the cable closest to the bore of the MRI scanner—should preferably be housed in a grounded nonferrous metal tube, such as aluminum. This would act as a shield to prevent electromagnetic interference between the electrical wires of the extended probe cable and the scanner of the MRI system.

As best shown in FIGS. 1-4, the housing 40 of the pulse oximetry probe 10 is overmolded onto the first and second fiber optic bundles 20 and 30 so that the emitter and detector portions 21 and 31 thereof are securely positioned diametrically opposite each other. The construction is such to place the emitter and detector portions 21 and 31 in near perfect alignment to get as much light as possible transferred from the emitter side to the detector side. The overmolding makes the alignment of the emitter and detector portions 21 and 31 largely impervious to movement of the finger when it is placed within the opening 41 defined by housing 40. When placed in opening 41, the finger is thus situated between the emitter and detector portions 21 and 31 that are overmolded by the upper and lower portions 42 and 43, respectively, of housing 40.

As best shown in FIG. 4, the housing 40 (also referred to as a grip) has its opening 41 ideally shaped to comfortably accommodate the finger or other body part. It is contemplated that grips of many different sizes will be offered to accommodate body parts, and even patients, of differing sizes. In that regard, the housing 40 is preferably formed from a flexible material. The shape of the grip, and its composition, would therefore be chosen so that the housing 40 would be adapted to fit onto the anatomical region of interest in such a manner as to provide a natural spring like fit.

Upon completion of the overmolding operation and the curing of housing 40, the emitter and detector portions 21 and 31 are securely positioned diametrically opposite each other a predetermined distance apart across the opening 41 into which the finger is designed to be inserted and held. The predetermined distance is dependent upon the size of the body part, and hence the size of the insert member. Flexibility of housing 40, of course, allows considerable stretching of the opening 41 to accommodate the body part.

FIGS. 6A-6F illustrate a fixture for making the probe. The fixture, generally designated 100, includes a base member 120, an opposing member 130, and the insert member 140 alluded to above. As best shown in FIGS. 6C-6F, the insert member 140 is intended for placement between the base and opposing members 120 and 130. The members 120, 130 and 140 together define a cavity of predetermined shape wherein the housing 40 of probe 10 is formable via introduction of a flowable material. The cavity, designated by reference numeral 150, is best understood via reference to FIGS. 6C and 6E. In that regard, section 141 of insert member 140 is the portion of fixture 100 around which the opening 41 of housing 40 is formed in cavity 150 via the method described below.

As best shown in FIGS. 6A-6C, the members further define two channels 160 and 170 for holding first and second fiber optic bundles 20 and 30, respectively, as housing 40 is overmolded thereabout so that the emitter portion 21 of first fiber optic bundle 20 and the detector portion 31 of second fiber optic bundle 30 are securely positioned diametrically opposite each other. Insert member 140 preferably includes a retaining bracket 145 and screws 148 with which to securely hold the fiber optic bundles 20 and 30 and aid in the assembly of channels 160 and 170. In addition, section 141 preferably has a hole 147 defined therein that acts as a slot on either side of insert member 140 into which the terminal ends of the emitter and detector portions 21 and 31 insert. Along with channels 160 and 170, these slots 147 enable the emitter and detector portions 21 and 31 to be held securely during formation of housing 40. Screws 128 may be used to secure insert member 140 onto base member 120. Screws 158 may also be used to secure opposing member 130 onto base member 120 and thus secure insert member 140 therebetween.

The flowable material may be selected preferably from any one or more of a number of rubber and/or elastomeric compounds including, but not limited to, silicone, fluorosilicone, urethane, polyurethane, polyethylene, and various propylene compounds. The flowable material is introduced into the cavity 150 via at least one conduit or runner defined by at least one of the base, opposing and insert members 120, 130 and 140. Two such conduits, 151 and 152, are shown in FIG. 6F by way of example. The housing 40 is thus formed via overmolding of the ends of fiber optic bundles 20 and 30. The overmolding of the fiber optic bundles 20 and 30 makes the alignment of the emitter and detector portions 21 and 31 largely impervious to movement of a body part when the body part is positioned within the opening 41 of housing 40 and between the emitter and detector portions 21 and 31 therein during use of the pulse oximetry probe 10.

The invention also provides a method of making the probe 10. The steps of the method are described below and are readily understood by reference to FIGS. 6A-6F. The steps include: (a) attaching a base member of a mold to a stationary platen of a molding system; (b) attaching an opposing member of the mold to a movable platen of the molding system; (c) positioning a pair of fiber optic bundles on an insert member of the mold so that an emitter portion of a first of the fiber optic bundles and a detector portion of a second of the fiber optic bundles are positioned diametrically opposite each other a predetermined distance apart; (d) placing the insert member on which the emitter and detector portions have been positioned upon the base member of the mold; (e) pressing the opposing member onto the insert member via the movable and stationary platens of the molding system, with the members together defining a cavity of predetermined shape wherein a housing of the probe is formable via introduction of a flowable material therein; and (f) introducing the flowable material into the cavity thereby overmolding the fiber optic bundles therein and forming the housing thereabout so that the emitter and detector portions are securely positioned diametrically opposite each other across an opening defined by the housing. The overmolding makes the alignment of the emitter and detector portions largely impervious to movement of a body part when the body part is positioned within the opening of the housing and thus between the emitter and detector portions therein during use of the probe. The predetermined distance is dependent upon the size of the body part, and hence the size of the insert member and the cavity defined therewith. Flexibility of the housing, of course, allows considerable stretching of the opening to accommodate the body part.

The molding system employed by the method is intended to encompasses a wide variety of molding systems including reaction injection molding (RIM) and liquid injection molding (LIM) systems as well as other injection molding techniques.

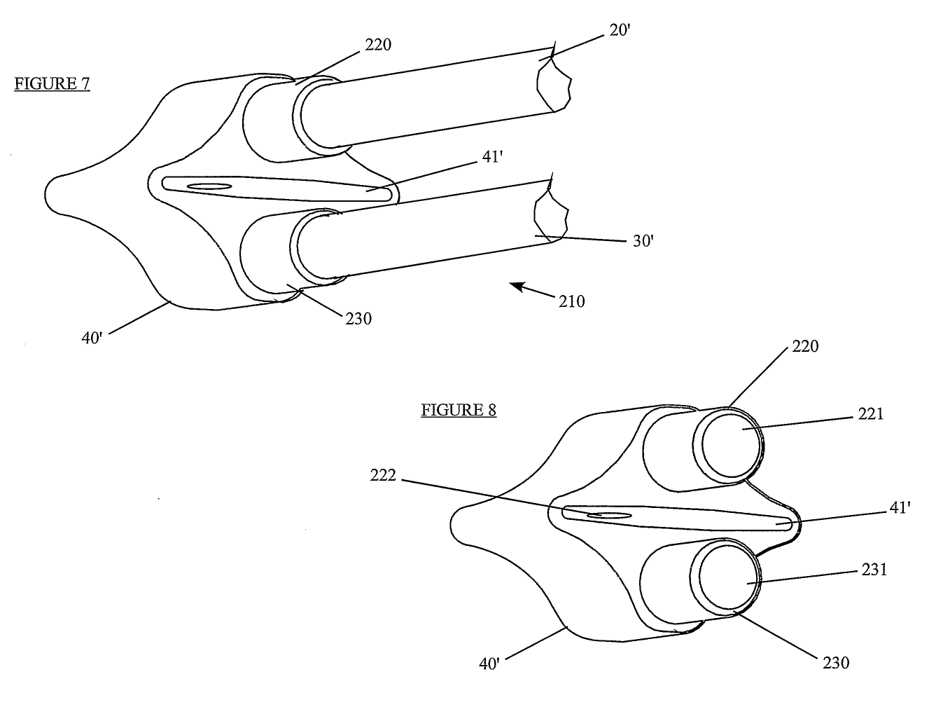

FIGS. 7-8 illustrate the pulse oximetry probe made according to a second embodiment of the invention. In this presently preferred embodiment, the probe, generally designated 210, includes two fiber optic bundles 20′ and 30′, two subhousings 220 and 230, and a housing 40′. The fiber optic bundles 20′ and 30′ are preferably identical to those disclosed in connection with the first embodiment inclusive of the emitter and detector portions. The subhousings, however, are new to this particular embodiment. Each subhousing 220 and 230 defines an internal conduit into which an end of its respective fiber optic bundle is inserted and retained as best shown in FIGS. 8-10.

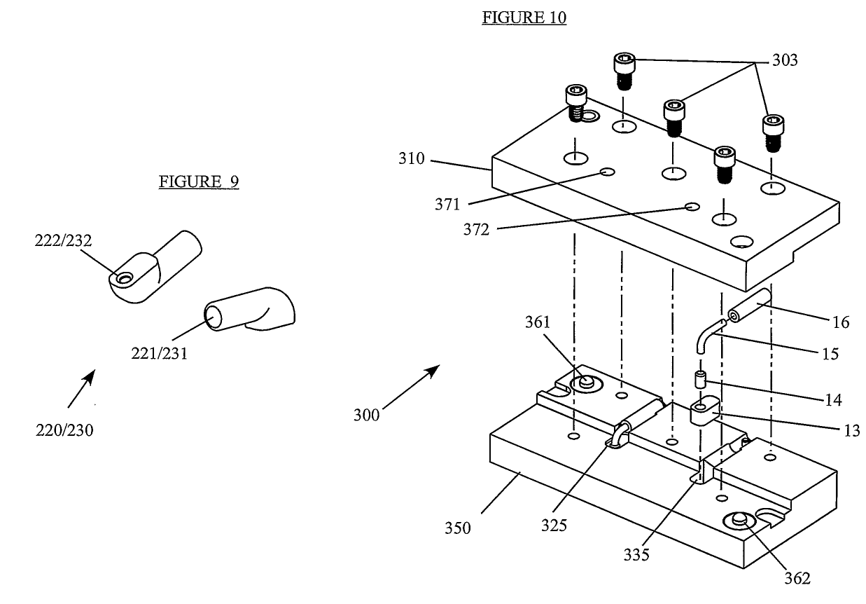

Subhousings 220 and 230 make it possible to hold more securely the emitter and detector portions during the manufacture of probe 210 than does the method of assembly disclosed in connection with probe 10 of the first embodiment. As shown in FIGS. 8 and 9, the internal conduit 221 of subhousing 220 is designed to accommodate insertion of the end of fiber optic bundle 20′ so that its emitter portion is securely held therein with its polished face aimed outward from aperture 222. Similarly, the internal conduit 231 of subhousing 230 accommodates insertion of the end of fiber optic bundle 30′ so that its detector portion is securely held therein with its polished face aimed outward from aperture 232.

Subhousings 220 and 230 can be formed, for example, using the subfixture 300 shown in FIG. 10. Subfixture 300 includes a top member 310 and a bottom member 350 that when mated together define two chambers 325 and 335 (shown in part) for use in molding the two subhousings. Within each chamber, one subhousing can be produced using a casing piece 13 and a cylindrical bushing 14 along with a tubular insert 15 and a sheath 16. Specifically, the retaining bushing 14 is designed to fit snugly into a hole defined in casing piece 13. By its curved end, the tubular insert 15 is then inserted into the bore of retaining bushing 14. Sheath 16 is used to cover the other, longer end of tubular insert 15. With the resulting assemblies placed within the chambers (see, e.g., chamber 325), the top member 310 is then aligned onto bottom member 350 via tabs 361 and 362 and then secured thereto via screws 303. A soft silicone or other suitable compound can then be injected into either of molding conduits 371 or 372, with the other molding conduit acting as the vent for subfixture 300. Upon injection of the elastomeric compound into the mold, the subhousings 220 and 230 form about the assemblies within chambers 325 and 335, respectively, inside subfixture 300.

Upon disassembly of top member 310 from bottom member 350, the molded assemblies can then be removed from chambers 325 and 335. The tubular insert 15 and sheath 16 can then be readily extracted from each subhousing due to its elastic properties. The resulting subhousings are best shown in FIG. 9. Each subhousing is then ready to receive its corresponding fiber optic bundle. Specifically, the first fiber optic bundle 20′ by its curved end is inserted into conduit 221 of subhousing 220 along with a measured amount of adhesive to secure the periphery of the bundle to the inner wall of the subhousing. The emitter portion of fiber optic bundle 20′ should be positioned so that its face is aimed outward from aperture 222. Likewise, the second fiber optic bundle 30′ by its curved end is inserted into, and secured by adhesive, within conduit 231 of subhousing 230. The detector portion of fiber optic bundle 30′ should be positioned so that its face is aimed outward from aperture 232. An optically clear silicone, epoxy or other suitable material is then preferably applied over the aperture of each subhousing as further protection for the emitter and detector portions.

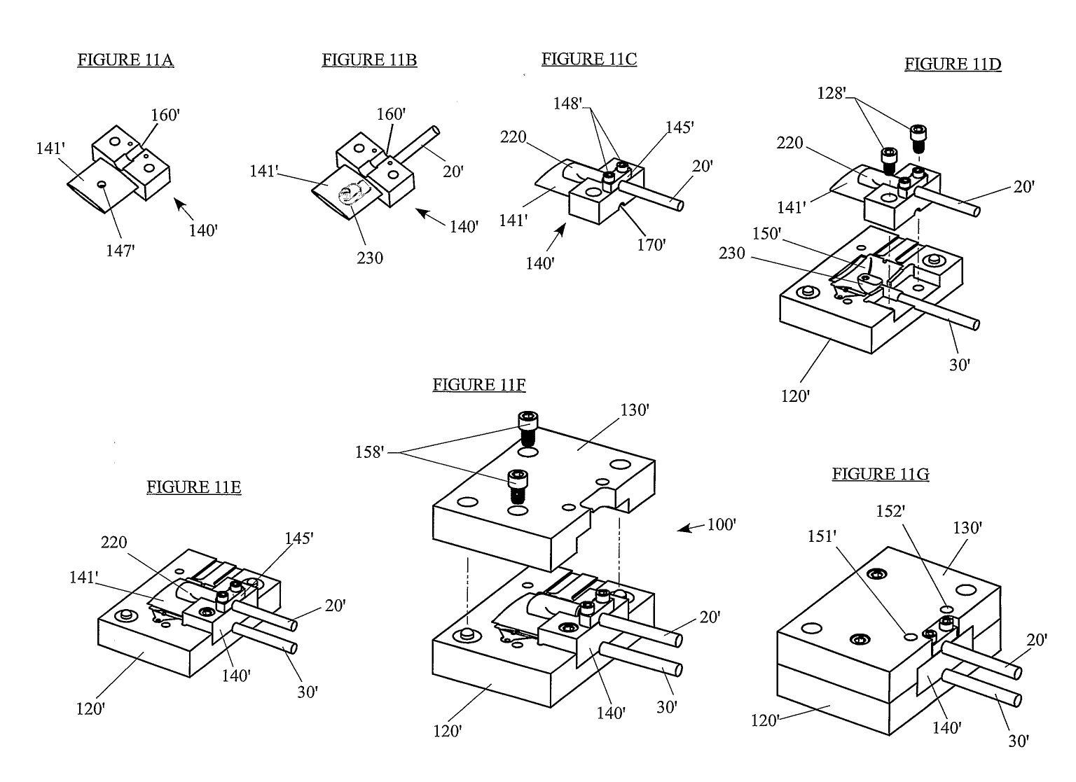

FIGS. 11A-11G also illustrate a fixture for making pulse oximetry probe 210. In this presently preferred embodiment, the fixture, generally designated 100′, is nearly identical to that disclosed in the first embodiment, i.e., fixture 100. The differences between fixtures 100 and 100′ lie mainly in the size and shape of cavity 150′ in the latter. The cavity 150′ is, of course, defined by the base, opposing and insert members 120′, 130′ and 140′, and it must accommodate subhousings 220 and 230 during the formation of housing 40′ via the overmolding process.

As best shown in FIGS. 11D-11G, insert member 140′ is intended for placement between the base and opposing members 120′ and 130′. The members 120′, 130′ and 140′ together define the cavity 150′ of predetermined shape wherein housing 40′ of probe 210 is formable via introduction of the flowable material. The cavity 150′ is best understood via reference to FIGS. 11D and 11F. Upon assembly of fixture 100′, section 141′ of insert member 140′ lies within cavity 150′, and is the portion of the fixture around which the opening 41′ of housing 40′ is formed via the method described below.

As best shown in FIGS. 11A-11C, the members further define channels 160′ and 170′ for holding the fiber optic bundles 20′ and 30′, respectively, as housing 40′ is molded over subhousings 220 and 230 so that the emitter portion of fiber optic bundle 20′ and the detector portion of fiber optic bundle 30′ are securely positioned diametrically opposite each other. Insert member 140′ preferably includes bracket 145′ and screws 148′ with which to securely retain the fiber optic bundles and aid in the assembly of channels 160′ and 170′. In addition, section 141′ preferably has a hole 147′ defined therein that acts as a guide on either side of insert member 140′ so as to aid in the alignment of the emitter and detector portions. Screws 128′ may be used to secure insert member 140′ onto base member 120′. Similarly, screws 158′ can be used to secure opposing member 130′ onto base member 120′ and thus secure insert member 140′ therebetween.

The flowable material is introduced into cavity 150′ via at least one conduit. Two such conduits, 151′ and 152′, are shown in FIG. 11G by way of example. The housing 40′ is thus formed via overmolding of the subhousings 220 and 230. As explained above, the overmolding of the fiber optic bundles 20′ and 30′ makes the alignment of the emitter and detector portions largely impervious to movement of a body part when the body part is positioned within the opening 41′ of housing 40′ and between the emitter and detector portions therein during use of the pulse oximetry probe 210.

The invention also provides a method of making the probe 210. The steps of the method are described below and are readily understood by reference to FIGS. 11A-11G. The steps include: (a) inserting an emitter portion of a first fiber optic bundle into an emitter subhousing; (b) inserting a detector portion of a second fiber optic bundle into a detector subhousing; (c) positioning the emitter and detector subhousings on an insert member of a mold so that the emitter portion of the first fiber optic bundle and the detector portion of the second fiber optic bundle are positioned diametrically opposite each other a predetermined distance apart; (d) placing the insert member on which the emitter and detector subhousings have been positioned upon a base member of the mold; (e) placing an opposing member of the mold onto the insert member, with the base, insert and opposing members together defining a cavity of predetermined shape wherein a housing of the probe is formable via introduction of a flowable material therein; and (f) introducing the flowable material into the cavity to at least partially overmold the emitter and detector subhousings therein thereby forming the housing thereabout so that the emitter and detector portions are securely positioned diametrically opposite each other across an opening defined by the housing so that an alignment of the emitter and detector portions is made largely impervious to movement of a body part when the body part is positioned within the opening of the housing during use of the probe.

It should be apparent that the base, insert and opposing members of fixture 100′ may be used to manufacture probe 210 with the aid of the stationary and movable platens of a molding system. This is described above in connection with the method of producing the probe 10 of the first embodiment. As with fixture 100 of the first embodiment, fixture 100′ of this presently preferred embodiment may be used to produce the probe of the invention with or without resort to such a molding system. Use of such platens, however, enables the manufacture of the probes 10 and 210 to be automated, particularly when an array of fixtures is employed simultaneously. Alternatively, one large fixture capable of producing a larger number of probes simultaneously may be used.

From the foregoing, it should be understood that the invention provides a reusable and easy to use pulse oximetry probe that can be produced and fitted for patients of different sizes and sites of use. It provides a robust design that substantially ensures proper alignment of the emitter and detector portions, particularly with probes intended for use with neonatal patients. Because the housing is overmolded onto the emitter and detector portions of the fiber optic bundles, the emitter and detector portions are not able to pop out of the coupling members as is all too common with prior art probes. The probes, fixtures and methods of the invention thus solve the problem of misalignment of emitter and detector portions, and therein save medical personnel the time that they heretofore previously spent on tracking down the source(s) of erroneous SpO2 readings.

Several embodiments and related aspects for carrying out the invention have been set forth in detail according to the Patent Act. Persons of ordinary skill in the art to which this invention pertains may nevertheless recognize alternative ways of practicing the invention without departing from the spirit of the following claims. Consequently, all changes and variations that fall within the literal meaning, and range of equivalency, of the claims are to be embraced within their scope. Persons of such skill will also recognize that the scope of the invention is indicated by the claims rather than by any particular example or embodiment discussed in the foregoing description.

Accordingly, to promote the progress of science and the useful arts, the inventor(s) hereby secure by Letters Patent exclusive rights to all subject matter embraced by the following claims for the time prescribed by the Patent Act.

What is claimed is:

1. A probe for use with a pulse oximeter apparatus, the probe comprising:
(a) a first fiber optic bundle having an emitter portion at one end thereof, said first fiber optic bundle for conducting light from a source thereof to said emitter portion from which the light is transmitted for transillumination through a body part;
(b) a second fiber optic bundle having a detector portion at one end thereof, said second fiber optic bundle for conducting the transilluminated light incident upon said detector portion to said pulse oximeter apparatus; and
(c) a housing wherein said emitter and said detector portions of said first and said second fiber optic bundles, respectively, are overmolded so that said emitter and said detector portions are securely positioned diametrically opposite each other across an opening defined by said housing thereby making an alignment of said emitter and said detector portions largely impervious to movement of the body part when placed within said opening of said housing and thus between said emitter and said detector portions therein.

2. The probe claimed in claim 1 wherein said emitter and said detector portions are securely positioned diametrically opposite each other a predetermined distance apart, with said predetermined distance being dependent upon at least a size of said insert member.

3. The probe claimed in claim 1 wherein said emitter and said detector portions are securely positioned diametrically opposite each other a predetermined distance apart, with said predetermined distance being dependent upon at least a size of the body part.

4. The probe claimed in claim 1 wherein the body part said opening is shaped to accommodate includes at least one of a finger, an earlobe, and a nose.

5. The probe claimed in claim 1 wherein said housing is made of a flexible material.

6. A fixture for making a probe for a pulse oximeter apparatus, the fixture comprising:
(a) a base member;
(b) an opposing member; and
(c) an insert member for placement between said base and said opposing members and therewith defining a cavity of predetermined shape wherein a housing of the probe is formable via introduction of a flowable material therein via at least one conduit defined by at least one of said members, said members further defining two channels for holding first and second fiber optic bundles, respectively, as said housing is overmolded thereabout so that an emitter portion of said first fiber optic bundle and a detector portion of said second fiber optic bundle are securely positioned diametrically opposite each other across an opening defined by said housing thereby making an alignment of said emitter and said detector portions largely impervious to movement of a body part when the body part is positioned within said opening of said housing and thus between said emitter and said detector portions therein during use of the probe.

7. The fixture claimed in claim 6 wherein said emitter and said detector portions are securely positioned diametrically opposite each other a predetermined distance apart, with said predetermined distance being dependent upon at least a size of said insert member.

8. The fixture claimed in claim 6 wherein said emitter and said detector portions are securely positioned diametrically opposite each other a predetermined distance apart, with said predetermined distance being dependent upon at least a size of the body part.

9. The fixture claimed in claim 6 wherein:
(a) said base member of said mold is adapted for connection to a stationary platen of a molding system; and
(b) said opposing member of said mold is adapted for connection to a movable platen of said molding system;
with said molding system enabling said movable platen to close upon said insert member situated on said base member so that said members are pressed tightly together as the flowable material is introduced into said cavity to form said housing therein.

10. The fixture claimed in claim 6 wherein the body part said opening is shaped to accommodate includes at least one of a finger, an earlobe, and a nose.

11. The fixture claimed in claim 6 wherein the flowable material of said housing is flexible upon curing.

12. A method of making a probe for use with a pulse oximeter apparatus, the method comprising the steps of:
(a) positioning a pair of fiber optic bundles on an insert member of a mold so that an emitter portion of a first of said fiber optic bundles and a detector portion of a second of said fiber optic bundles are positioned diametrically opposite each other a predetermined distance apart;
(b) placing said insert member on which said emitter and said detector portions of said fiber optic bundles have been positioned upon a base member of said mold;
(c) placing an opposing member of said mold onto said insert member, with said base, said insert and said opposing members together defining a cavity of predetermined shape wherein a housing of said probe is formable via introduction of a flowable material therein; and
(d) introducing the flowable material into said cavity thereby overmolding said fiber optic bundles therein and forming said housing thereabout so that said emitter portion and said detector portion are securely positioned diametrically opposite each other across an opening defined by said housing so that an alignment of said emitter and said detector portions is made largely impervious to movement of a body part when the body part is positioned within said opening of said housing during use of the probe.

13. The method claimed in claim 12 wherein said predetermined distance is dependent upon at least a size of said insert member.

14. The method claimed in claim 12 wherein said predetermined distance is dependent upon at least a size of the body part.

15. The method claimed in claim 12 wherein the flowable material is introduced into said cavity via at least one conduit defined by at least one of said base member, said insert member and said opposing member.

16. The method claimed in claim 12 wherein the body part said housing is shaped to accommodate includes at least one of a finger, an earlobe, and a nose.

17. The method claimed in claim 12 wherein the flowable material of said housing is flexible upon curing.

18. A method of making a probe for use with a pulse oximeter apparatus, the method comprising the steps of:
(a) attaching a base member of a mold to a stationary platen of a molding system;
(b) attaching an opposing member of said mold to a movable platen of said molding system;
(c) positioning a pair of fiber optic bundles on an insert member of said mold so that an emitter portion of a first of said fiber optic bundles and a detector portion of a second of said fiber optic bundles are positioned diametrically opposite each other a predetermined distance apart;
(d) placing said insert member on which said emitter and said detector portions of said fiber optic bundles have been positioned upon said base member of said mold;
(e) pressing said opposing member onto said insert member via said movable and said stationary platens with said base, said insert and said opposing members together defining a cavity of predetermined shape wherein a housing of said probe is formable via introduction of a flowable material therein; and
(f) introducing the flowable material into said cavity thereby overmolding said fiber optic bundles therein and forming said housing thereabout so that said emitter portion and said detector portion are securely positioned diametrically opposite each other across an opening defined by said housing so that an alignment of said emitter and said detector portions is made largely impervious to movement of a body part when the body part is positioned within said opening and thus between said emitter and said detector portions therein during use of the probe.

19. The method claimed in claim 18 wherein said predetermined distance is dependent upon at least a size of said insert member.

20. The method claimed in claim 18 wherein said predetermined distance is dependent upon at least a size of the body part.

21. The method claimed in claim 18 wherein the flowable material is introduced into said cavity via at least one conduit defined by at least one of said base member, said insert member and said opposing member.

22. The method claimed in claim 18 wherein the body part said housing is shaped to accommodate includes at least one of a finger, an earlobe, and a nose.

23. The method claimed in claim 18 wherein the flowable material of said housing is flexible upon curing.

24. A probe for use with a pulse oximeter apparatus, the probe comprising:
(a) a first fiber optic bundle having an emitter portion at one end thereof, said first fiber optic bundle for conducting light from a source thereof to said emitter portion from which the light is transmitted for transillumination through a body part;
(b) a second fiber optic bundle having a detector portion at one end thereof, said second fiber optic bundle for conducting the transilluminated light incident upon said detector portion to said pulse oximeter apparatus;
(c) an emitter subhousing into which said emitter portion of said first fiber optic bundle is inserted;
(d) a detector subhousing into which said detector portion of said second fiber optic bundle is inserted; and
(e) a housing wherein said emitter and said detector subhousings are overmolded so that said emitter and said detector portions inserted therein, respectively, are securely positioned diametrically opposite each other across an opening defined by said housing thereby making an alignment of said emitter and said detector portions largely impervious to movement of the body part when placed within said opening and between said emitter and said detector portions therein.

25. A fixture for making a probe for a pulse oximeter apparatus, the fixture comprising:
(a) a base member;
(b) an opposing member; and
(c) an insert member for placement between said base and said opposing members and therewith defining a cavity of predetermined shape wherein a housing of the probe is formable via introduction of a flowable material therein via at least one conduit defined by at least one of said members, said members further defining two channels for holding first and second fiber optic bundles, respectively, as said housing is overmolded about both an emitter subhousing and a detector subhousing into which an emitter portion of said first fiber optic bundle and a detector portion of said second fiber optic bundle have been respectively inserted so that said emitter and said detector portions are securely positioned diametrically opposite each other across an opening defined by said housing thereby making an alignment of said emitter and said detector portions largely impervious to movement of a body part when the body part is positioned within said opening and between said emitter and said detector portions therein during use of the probe.

26. A method of making a probe for use with a pulse oximeter apparatus, the method comprising the steps of:
(a) inserting an emitter portion of a first fiber optic bundle into an emitter subhousing;
(b) inserting a detector portion of a second fiber optic bundle into a detector subhousing;
(c) positioning said emitter and said detector subhousings on an insert member of a mold so that said emitter portion of said first fiber optic bundle and said detector portion of said second fiber optic bundle are positioned diametrically opposite each other a predetermined distance apart;
(d) placing said insert member on which said emitter and said detector subhousings have been positioned upon a base member of said mold;
(e) placing an opposing member of said mold onto said insert member, with said base, said insert and said opposing members together defining a cavity of predetermined shape wherein a housing of said probe is formable via introduction of a flowable material therein; and
(f) introducing the flowable material into said cavity to at least partially overmold said emitter and said detector subhousings therein thereby forming said housing thereabout so that said emitter and said detector portions are securely positioned diametrically opposite each other across an opening defined by said housing so that an alignment of said emitter and said detector portions is made largely impervious to movement of a body part when the body part is positioned within said opening of said housing during use of the probe.

27. A method of making a probe for use with a pulse oximeter apparatus, the method comprising the steps of:
(a) attaching a base member of a mold to a stationary platen of a molding system;
(b) attaching an opposing member of said mold to a movable platen of said molding system;
(c) inserting an emitter portion of a first fiber optic bundle into an emitter subhousing;
(d) inserting a detector portion of a second fiber optic bundle into a detector subhousing;
(e) positioning said emitter and said detector subhousings on an insert member of said mold so that said emitter portion of said first fiber optic bundle and said detector portion of said second fiber optic bundle are positioned diametrically opposite each other a predetermined distance apart;
(f) placing said insert member on which said emitter and said detector subhousings have been positioned upon said base member of said mold;
(g) pressing said opposing member onto said insert member via said movable and said stationary platens with said members together defining a cavity of predetermined shape wherein a housing of said probe is formable via introduction of a flowable material therein; and
(h) introducing the flowable material into said cavity to at least partially overmold said emitter and said detector subhousings therein thereby forming said housing thereabout so that said emitter and said detector portions are securely positioned diametrically opposite each other across an opening defined by said housing so that an alignment of said emitter and said detector portions is made largely impervious to movement of a body part when the body part is positioned within said opening of said housing and thus between said emitter and said detector portions therein during use of the probe.
